(19) United States
(12) Patent Application Publication
(10) Pub. No.: US 2002/0172707 A1
(43) Pub. Date: Nov. 21, 2002

(54) Ultra-nutraceuticals and toothbursh sanitizer (ozone)

(76) Inventor: Catherine Joyce, Chester, NH (US)

Correspondence Address: Catherine Joyce, 858 Candia Road, Chester, NH, 03036-4012, US

(21) Appl. No.: 09/848,590

(22) Filed: May 3, 2001

Related U.S. Application Data
(60) Provisional application No. 60/201,492, filed on May 3, 2000.

(51) Int. Cl. A61K 7/16; A61K 47/00

(52) U.S. Cl. 424/439

(57) ABSTRACT

New methods of manufacture, uses and applications for nutraceuticals, their compounds, extracts, enzymes, and special new properties, including, but not limited to, anthocyanidins, pro-anthocyanidins, dimers, polymers, crystalline proanthocyanadin, leucocyanin, leucodelphinin, flavornoids, and polyphenols, hereafter coined “ultra-nutraceuticals”.

These new methods, applications, properties and uses offer superior environmental, performance, medical and economic alternatives to chemical, synthetic and other natural-based wet/dry regular or concentrated compounds from herbs, vegetables, fruits and natural fibers. These ultra-nutraceuticals are desired in the manufacture of, but not limited to, pharmaceuticals for treatment and prevention of disease, medical: wound dressings, surgical gowns/drapes; film barriers: wet/dry soaps, cleaning solutions, sprays and surface coatings; deodorants/antiperspirants; nonwovens, including moist/dry sheets & towels, feminine hygiene, incontinence and diapers; cosmetics; personal care; animal bedding and pet litter; agricultural sprays; compositions of matter such as sponges, paper and molded pulp products, and meat/poultry/fruit trays.

New application for toothbrush sanitizer in the small appliance category. New appliance consists of an electric, hand-held toothbrush sanitizer fueled by ozone, which can be used with or without toothpaste. Ozone electric toothbrush removes the need for toothpaste as ozone kills 99.9% of all bacteria in mouth within two seconds. Ozone is a safe, natural antibacterial chemical as compared to Colgate's Total toothpaste which contains triclosan as an active ingredient for antimicrobial effectiveness. Sales of this electric toothbrush could erode global toothpaste sales affecting the markets of leading toothpaste manufacturers Colgate and Procter & Gamble.

### ULTRA-NUTRACEUTICALS

New growing, harvesting, processing and manufacturing methods of nutraceuticals will enhance present methods and compounds and offer new properties which will expand the number of new product applications from all natural resources. Consumers are becoming very sophisticated in the knowledge and utilization of nutraceuticals [herbal and dietary supplements]. They prefer “natural” resources be used whenever possible as these have fewer side effects.

### BACKGROUND

### Field of Invention

The following new uses and applications of nutraceuticals introduces new properties of odor control, inhibits bacterial growth, antimicrobial, antiseptic, antibiotic, anti-fungal, anti-inflammatory, triggers cellular toggle switch, cellular blocker, statin properties: lowers cholesterol, strengthens capillaries, suppresses appetite, improves mental and neural capacity, excitory neurotransmitter, besides their published existing science-based properties of dietary supplements, non-viral, antioxidant [anti-aging], and anti-adhesion properties on epithelial cells which prevent and treat urinary/bladder infections caused by e-coli and salmonella, et al.

### New Methods of Manufacture

Changes have been made in the growing, harvesting, processing, storage and delivery systems of ultra-nutraceuticals; pending full accounting of results.

### NEW USES AND APPLICATIONS

### Natural Anti-microbial Cleaners/Spray/Surface Coatings

Anti-bacterial liquid and solid cleaners contain active ingredients of Triclosan, Tricloban or Benzylkonium Chloride. This invention creates “natural” anti-bacterial cleaners, liquid/solid soaps, lotions, veggie-wash sprays [enhances P&G's new product “Fit” or offers competitive advantage to new entrants]. Natural antibacterial solutions support environmental concerns with little or no side effects. They are safe enough to be ingested, and are through dietary supplements, a $10 Billion dollar market. Natural molecules have only active ingredients, that is why they are so valuable to the pharmaceutical market.

Consumers are concerned with residue left from anti-bacterial chemicals on plates, glasses, utensils, which are ingested killing “good bacteria” and requiring consumers to buy expensive, probiotic products such as yogurt, containing live bacteria cultures to protect against/prevent colon disease, et al.

Natural anti-bacterial cleaners made from ultra-nutraceuticals would enhance products such as P&G's “Fit” because when added to “Fit”, they would extend the shelf life of the fruit or vegetables from 1-2 days-to weeks by creating an anti-bacteria film barrier which does not allow e-coli or salmonella, etc. to adhere to surface of produce; solution is completely harmless to human consumption. Otherwise, growers associations could initially wash produce and then spray produce with natural anti-bacterial solution, thus preventing e-coli and salmonella infections.

Surface Coatings

Ultra-nutraceuticals would offer “natural” antimicrobial and antifungal properties to new cleaning products. e.g., P&G's Swiffer disposable cloths, thus preventing bacterial growth on cloths, and offering additional, premium marketing claims. Consumers could feel confident they were not swishing germs around household. These new, durable surface coatings would add value to focus on new antibacterial products.

Pharmaceuticals

Ultra-Nutraceuticals can greatly benefit existing RX drugs by pairing with established FDA-approved drugs, reducing the actual dose of RX drug by 25-35±%, thus decreasing the instances of side effects and drug resistance, and quicker FDA approvals. These combinations would prove helpful to reducing RX costs to substantial populations, and would also be patentable and approved in a shorter timeline. Using nutraceuticals in RX formulas would reduce the costs of clinical studies and approval time, thus lowering the cost of bringing RX to market. RX delivery includes inhalation sprays, transdermal applications, lotions, ointments, ingestion by gum, intravenous, solid tablet forms, liquid or gel forms, injections, dietary supplements, candy, nutritional bars, and time-release delivery systems.

Natural ultra-nutraceuticals in pharmaceutical formulas can be help prevent or treat Heart Disease by lowering chlorestorol. Science-based clinical studies and research conducted lend a very solid basis for this application.

Cancer: ultra-nutraceuticals have the ability to trigger cellular toggle switch and may be helpful in inhibiting telomerase production in cancer cells and blocking them from dividing. Telomerase is present in 85-90% of all cancer cells.

AIDS: The method of aids cell replication is to “hook” onto healthy cells thus avoiding immune system responses. Natural ultra-nutraceuticals are preventive and do not allow viral cells to “hook”, thus assisting researchers in their quest for an aids cure and possible alternate treatments using these natural ultra-nutraceuticals.

Multiple Sclerosis; Muscular Distrophy: ultra-nutraceuticals offer potential for prevention and treatment for these diseases.

Natural ultra-nutraceuticals are beneficial to environment as no further depletion of rainforests are needed to find equivalent, naturally efficacious antifungal, antimicrobial, antibiotic, antiseptic, anti-inflammatory drugs. Pharmaceutical manufacturers do not need to ravage third-world rainforests and natural habitats.

Natural ultra-nutraceuticals can also pass through blood/brain barrier to improve mental and neural capacity, thus offering hope to Creuzfeldt-Jakob [CJD or “mad cow”]. Alzheimers, Epilepsy, Parkinson's, and migrane headaches [26Mil sufferers], Diabetes and insomniacs. Ultra-nutraceutical's ability to trigger cellular toggle switch may be helpful in preventing these diseases. CJD is working on chemical solutions to disease, but ultra-nutraceuticals offer “natural” drug opportunities to efficaciously transform prions back into harmless configurations to treat disease, or prevent cell transformation in the first place.

Personal Care

Hair Shampoo, Conditioners, shaving cream, cuticle creams. Cuticle conditions presuppose openings in the skin lining the nailbeds. Cuticle sufferers often experience pain due to these open lesions as immune cells are fighting bacteria inflammations at site. Natural ultra-nutraceuticals used in shampoos and conditioners will leave a protective film on hair shaft preventing bacteria buildup so claims may be made that hair remains cleaner, longer, affording consumers the option of avoiding daily washing, Ultra-nutraceuticals also offer advantages of aromatherapy as they have naturally pleasant scents.

Consumer giants such as P&G are refocusing attention on high-end hair care in its new Physique line, utilizing expensive new marketing initiatives. Natural ultra-nutraceuticals are also gender neutral and offer competitive advantages in hair care category with premium margins, and stylists will not be needed to demonstrate how to use these products, thus lowering the costs of marketing and manufacturing. Ultra-nutraceuticals are also “precision palm aid” and can be worked into the hair, and are naturally volumizing.

Ultra-nutraceuticals used in shaving cream and skin fresheners will offer natural antimicrobial properties on skin. Dental applications of natural ultra-nutraceuticals offer anti-inflammatory benefits to gingivitis sufferers in alleviating this condition in addition to providing a film coating to teeth and gums to prevent the adhesion of bacteria to gums and teeth. This natural application of ultra-nutraceuticals also offers odor control, thus reducing bad breath. By adding ultra-nutraceuticals to fluoride treatments, less tooth decay and gum disease would result.

Cosmetics

Acne treatments, moisturizers, lotions, creams, astringents. Natural ultra-nutraceuticals benefit cosmetics by applying a film layer to skin which discourages the growth of bacteria. Acne is caused by bacteria buildup and natural antimicrobial solutions can prevent or treat this serious skin condition.

Consumers prefer “natural” antimicrobial additives to their moisturizers, lotions, creams for their face, and especially their hands as this is the optimum method of bacterial contamination on all surfaces. Ultra-nutraceuticals also have astringent properties.

Deodorants/Antiperspirants

Aluminum chloride use is a known cancer risk and also the culprit responsible for fabric degradation. Consumers are warned to avoid deodorants/a-p's with aluminum chloride. The only “natural” deodorant product on the market is Tom's of Maine. Ultra-nutraceuticals used in deodorants and antiperspirants now can claim the reduction/elimination of underarm odor, without any cancer risk. This new product can be offered for a premium price, while lowering present deodorant/antiperspirant manufacturing costs.

For those consumers who prefer not to shave underarm or legs, ultra-nutraceuticals still offer them the benefits of odor control as its antimicrobial film properties coat the hair shaft, along with the underarm skin area, to reduce bacterial odor.

Nonwovens

Moist/Dry Towels, Feminine Hygiene, Incontinence & Diapers Ultra-nutraceuticals will prevent bacteria buildup which causes toxic shock syndrome in tampons/feminine pods; pressure sores and infections in incontinence pad users; and prevent diaper rash in babies.

Hygiene

Moist/Dry towels treated with natural ultra-nutraceutical formulations can offer antimicrobial properties without chemicals now, consumers' preference. These towels can be also be safely used for cleaning childrens and pets' toys which young children place in their mouths, without fear of chemical ingestion. Moist both tissue products can now be treated with ultra-nutraceutical formulations to prevent anal/perineal bacteria buildup and odor control.

There has never been a “natural”, science-based solution to bacteria buildup on tampons and feminine pads. Bacteria buildup causes bad odors associated with menstrual cycles and all consumers seek deodorizers and fresh smelling material alternatives, even one-way fluid cover sheets to solve this continual problem. Now these absorbent cores and cover sheets can be treated with ultra-nutraceuticals providing them the most effective and natural, science-based solution to odor control. This product lends itself to premium prices while eliminating the need for several odor-control layers.

Pressure sores(bedsores) are caused by enzymes/bacteria on skin breaking down the epithelial layers, leading to infections and expensive pharmaceutical solutions. Ultra-nutraceuticals incorporated into skin lotions, moisturizers, creams and body sprays could prevent these outbreaks. Patients still need to be turned, but these new products would help prevent rapid epithelial cell deterioration.

Diaper Rash

Absorbent cores and sheets are a source of urine and e-coli bacteria buildup in diapers, which causes diaper rash. P&G introduced Rashguard offering petrolatum strips and stearate, a skin conditioner to alleviate this problem. Natural ultra-nutraceuticals offer diaper cores, sheets and diaper tape, the benefits of a film barrier protecting skin from irritants which breakdown epithelial cells and lead to bacterial inflammation of site causing diaper rash. Antimicrobial protection on diaper tapes prevents dermatitis, an erythemic skin condition affecting the hip area under these tapes. Usual dermatologic treatment is by topical anticandidal creams and hydrocortizone, but natural ultra-nutraceuticals can prevent this condition from occuring under tapes. Staphylococcal diaper infections occur in nurseries/childcare facilities and become epidemic and are caused by carriers of toxin-producing bacterial strains. It is suffice to say that prevention of bacterial growth is key factor to avoiding diaper rash, and this is the strength of ultra-nutraceutical film barriers.

Agricultural Sprays

Natural anti-microbial properties in ultra-nutraceuticals offer Anti-fungal properties to pestilence of agricultural crops. Specifically, the citrus industry is constantly plagued by airborne citrus-canker outbreaks which destroy huge groves. Citrus-canker is a highly contagious, airborne, bacterial disease which attacks citrus fruit groves. We can prevent this disease by spraying all crops with ultra-nutraceuticals which create a film barrier on fruit to prevent bacteria from adhering. This could result in millions of dollars of savings in crop, pesticides. etc. These ultra-nutraceuticals cause no harm to humans or animals.

Medical

Wound dressings, bandaids, surgical gowns/drapes and operating room equipment, including air-ducts and vents. The anti-microbial properties in ultra-nutraceutical formulations prevent sterile surfaces from growing bacteria. Ultra-nutraceuticals form a tough film on surfaces and is not easily removed. Operating room utensils can be sterilized and then sprayed with natural ultra-nutraceutical formulation keeping bacteria from adhering to their surfaces. Many hospitals and institutions have continual problems with ventilation systems building up mold, fungus, etc. causing bacterial outbreaks [legionnaires disease]. Brigham & Womens Hospital in Boston had to close down several operating rooms and a newborn unit to facilitate a complete cleaning and renovation of these areas, and they still have problems as it never goes away.

Animal Bedding and Pet Litter

The Holy Grail of animal bedding and pet litter is to manufacture a product which does not encourage infection in livestock. Ultra-nutraceuticals in both these products could be manufactured into the product or sprayed as a surface coating. Premium prices can be charged by using ultra-nutraceuticals in these products so companies can make these science-based antimicrobial claims.

Ralston Purina just introduced dog litter which will increase volume in this market when competitors respond. Ultra-nutraceuticals added to pet litter will prevent bacteria from growing, thus keeping litter, fresher/longer. Odor control is a very big problem in this market and no “natural” alternative exists for discouraging growth of bacteria, only natural masking properties of baking soda.

Ultra-nutraceuticals offer science-based antimicrobial surface coatings for pet toys which after being knawed by pets, are later picked up by children and placed in their mouths. Ultra-nutraceuticals can end this cycle of infection.

Compositions of Matter

Sponges, Paper, Moulded Pulp Products, Meat/Poultry/Fruit Trays & Paks are just a few of the products which could benefit from the addition of ultra-nutraceuticals. Sponges are bacteria-breeders and new antibacterial coatings are trying to address this problem. Ultra-nutraceuticals can be formed within the foam composition creating a lasting antimicrobial film barrier to which no bacteria will adhere. This solves a longstanding problem in that industry.

Moulded pulp products consist mostly of egg cartons and food travel paks. New regulations force the moulded pulp industry to continually refrigerate filled egg cartons so as not to encourage the growth of salmonella through shell breakage before reaching the consumer. Ultra-nutraceuticals formulated into moulded pulp products would discourage the formation of salmonella on egg cartons.

Meat/Poultry/Fruit Trays & Paks also undergo strict regulations as to marketing claims. The industry is constantly seeking an antimicrobial tray which can make valid science-based claims as to not promoting the growth of bacteria [e-coli, salmonella] in meat, poultry and fruit. Most product solutions still do not allow these claims to be made and the cost of new materials is prohibitive. Moulded pulp manufacturers would love to be able to compete with foam's tray counterpart in this market. Foam manufacturers would love to be able to make this science-based claim that bacterial growth is not encouraged on its surfaces. Moulded pulp and foam compositions can be made using ultra-nutraceuticals at lower costs than any alternative by either formulating into the composition or spraying the surface.

Second Invention:

| Subject: | Toothbrush Sanitizer |
|---|---|
| Inventor: | Catherine Joyce |
| Date: | May 3, 2001 |
| Specifications: | |

### BACKGROUND OF INVENTION

Electric toothbrushes hove numerous features important to dental and gum hygiene. There are also a number of toothbrush sanitizers which have been patented, the most recent one by Cyclopss being jointly marketed by Procter & Gamble and Otres, Inc., along with Crest toothpaste.

An ozone toothbrush sanitizer offers the best of both worlds. It both cleans the brush of 99.8% of all bacteria present in the brush and when inserted in the mouth, kills 99.8% of all oral bacteria within 2-3 seconds of brushing. This invention will reduce the global demand for toothpaste, in all but third-world countries.

An ozone toothbrush & sanitizer is a compact small, handheld appliance; ozone is a naturally-occuring chemical safe to use in oral applications. I would recommend this device to children 12 years and older. An ozone electric toothbrush would yield a premium price in the market.

Ozone has been used for water sanitization, cooling towers and laundry operations for many years. Over the past ten years, this technology has incurred several setbacks in the institutional laundry industry, particularly Cyclopss' and TrioClean's institutional laundry systems which were required to install a safety monitoring system, but with an ozone unit this small, there should be no restrictions or deterrents to market aceptance.

This Ozone toothbrush and sanitizer is different from Cyclopss' toothbrush sanitizer as it is one, handheld combinant unit, not a separate unit used exclusively for sanitizing toothbrushes in a singular unit as the Cyclopss model. Ozone in and of itself is the best detergent known to the laundry industry and can be used with hydrogen peroxide without loss of active O₃ chemical ingredient. This Ozone toothbrush represents the new uses and dental applications for antimicrobial [99.8%], tooth “whitener” and “dental placque remover” and “treatment of gingivitis”. This appliance may be used with Mentodent's toothpaste system as it contains peroxide, but not with chlorine or chlorinated water. All chlorinated water will hove to be filtered as chlorine negates the antimicrobial benefits of Ozone. This application should also be true for Cyclopss' toothbrush sanitizer—the water has to be cold and they must filter the water to remove chlorine.

What I claim as my invention re nutraceuticals are:

I claim:

1. Pharmaceuticals be paired with nutraceuticals forming a new class of patentable drugs and a method for treating Heart Disease, Atherosclerosis, Alzheimer, Diabetes, Cancer, Aids, Multiple Sclerosis, Multiple Distrophy, Epilepsy, Creuzfeldt-Jakob, Insomnia and Migranes.

2. Specific Nutraceutical formulas contain new properties of odor control, anti-microbial, antiseptic, anti-fungal, antibiotic, anti-inflammatory, anti-viral and act as a trigger mechanism for cellular reactions, cellular block, excitory neurotransmitter and film barrier to bacterial or placque formation.

3. Specific Nutraceutical compounds are natural antibacterial formulations for use and application in pharmaceuticals; nonwovens; hygiene; animal bedding & pet litter;

4. A method and biologically active compounds for the purpose of interfering with viral and non-viral microbial tissue or cell adhesion to a body tissue, a surface or to inanimate surface(s) comprising topical, internal or manufacture administering of ultra-nutraceutical formulations.
